(19) United States
(12) Patent Application Publication
(10) Pub. No.: US 2023/0323044 A1
(43) Pub. Date: Oct. 12, 2023

(54) SYNTHETIC METAL SYSTEM

(71) Applicant: ENSEN PTY LTD, Norman Park, Queensland (AU)

(72) Inventor: David Nommensen, Norman Park (AU)

(21) Appl. No.: 18/020,081

(22) PCT Filed: Aug. 6, 2021
(86) PCT No.: PCT/AU2021/050865

§ 371 Date: Feb. 6, 2023

(30) Foreign Application Priority Data
Aug. 6, 2020 (AU) 2020902760
Nov. 10, 2020 (AU) 2020904115

(51) Int. Cl. C08J 5/04; B29C 70/68

(52) U.S. Cl. CPC C08J 5/042; B29C 70/682; B29K 2075/00; C08J 2375/04

(57) ABSTRACT

A synthetic metal system comprising a frame member comprising a cellular structure including a plurality of openings; and a matrix material comprising a polymeric material, wherein the matrix material is configured to at least partially penetrate one or more of the plurality of openings in the frame member such that the frame member is at least partially encased within the matrix material.

### TECHNICAL FIELD

The present invention relates to a synthetic metal system. In particular, the present invention relates to a synthetic metal system that may replace an existing component of an article or device without affecting the purpose or operation of that component within the article or device and provide the article or device with a structural benefit.

### BACKGROUND

Metal and metal alloys have long been used in many products and systems due to the fact that many metals have advantageous qualities such as strength, rigidity, durability and so on.

However, the use of metals is not without its drawbacks. For instance, many metals are prone to corrosion when damaged or exposed to the elements. Similarly, metal components placed in abutment with one another can suffer from metal-on-metal wear. Further, in some situations (such as in surgical implants or jewellery) metal components can cause allergic reactions in users. In addition, some metals (such as lead) are harmful to the environment and the weight of some metals makes them unsuitable for some applications.

Globally, the cost of metal corrosion is estimated to be US$2.5 trillion. As a result, significant monetary savings stand to be made from utilising corrosion control practices. Many corrosion control practices are known. One of these is the use of galvanic anodes (also called sacrificial anodes), which are fabricated from a material having a more negative reduction potential than the material of the structure with which they are associated. The difference in potential between the galvanic anode and the material of the structure results in the corrosion of the galvanic anode in preference to the structure.

However, the drawback of traditional galvanic cathodes (or indeed most other corrosion control techniques) is that they provide no structural benefit to the structure to which they are applied.

Metal is commonly used to provide structure to softer materials due to its high strength. For instance, titanium and titanium alloys have been used for implants and prosthetic devices because they are strong, light weight, durable and biocompatible.

However, it has been reported that in certain applications, metal on metal wear may release trace amounts of metal particles into the tissues surrounding the implant causing soft tissue damage and implants containing titanium have also been associated with metal hypersensitivity reactions. While hypoallergenic implants (such as ceramic implants) are available, these are more expensive than titanium implants and not as strong.

Metal is also used to fabricate some devices, such as projectiles, as its high density enables the projectile to retain velocity.

However, metal-on-metal wear due to the action of the projectile on the bore of the gun may form potentially hazardous metal particles and dust. In addition, as lead, copper and tin are commonly used in the fabrication of projectiles discarded spent casings and bullets may cause environmental poisoning.

It will be clearly understood that, if a prior art publication is referred to herein, this reference does not constitute an admission that the publication forms part of the common general knowledge in the art in Australia or in any other country.

### SUMMARY OF INVENTION

Embodiments of the present invention provide a synthetic metal system, which may at least partially address one or more of the problems or deficiencies mentioned above or which may provide the public with a useful or commercial choice.

With the foregoing in view, the present invention in one form, resides broadly in a synthetic metal system comprising:
a frame member comprising a cellular structure including a plurality of openings; and
a matrix material comprising a polymeric material,

wherein the matrix material is configured to at least partially penetrate one or more of the plurality of openings in the frame member such that the frame member is at least partially encased within the matrix material.

The present invention provides numerous advantages over the prior art. For instance, the present invention may provide improved control of the manufacture of a device where the desired properties of the synthetic metal system (such as conductivity, strength, density, rigidity, etc) are to be located in specific regions of the synthetic metal system.

For instance, the present invention may provide a device with improved electrical or thermal conductivity by replacing one or more existing components of the device and without affecting the purpose or operation of that component within the device. In addition, the present invention, allows a user to optimise the conductivity of the device based upon the conditions in which the device is being operated. For instance, in the mining industry, the synthetic metal system may provide a device with cathodic protection, providing inexpensive and simple means of corrosion control caused by electrolysis in well fluids whilst reducing reliance on liquid chemical inhibitors and thereby potential environmental hazards (such as spillage, water contamination, or the like).

For instance, in the medical industry, the present invention may provide an implant or prosthetic which is less prone to wear and less likely to cause metal hypersensitivity reactions.

For instance, in the engineering of sports equipment, the present invention may improve control of the distribution of density or specific gravity of the layers of a ball and enable a manufacturer to design a ball having specific design criteria (such as weight, dimensions, initial velocity, etc.). In addition, a resiliently deformable synthetic metal system may improve the coefficient or restitution of a ball increasing the moment of inertia and providing a more stable and predictable flight.

For instance, in the engineering of projectiles, the present invention may enable the reduction in lead and other metals traditionally used in ammunition which prevents metal-on-metal contact in the bore of the gun, reducing abrasion and debris and subsequently extending barrel life and enabling cooler operating temperatures during firing. In addition, a synthetic metal system projectile may have less perceived recoil compared to conventional projectiles.

The frame member may be of any suitable size, shape or configuration. However, it will be understood that the size, shape and configuration of the frame member may vary depending on the desired physical properties of the synthetic metal system and the use of the synthetic metal system. In some embodiments of the invention, the frame member may be the same size, shape, or configuration of the synthetic metal system, or a different size, shape, or configuration.

In a preferred embodiment of the invention, the frame member may be substantially the same size, shape, and configuration as the synthetic metal system. In this instance, it is envisaged that the frame member may substantially define the shape of the synthetic metal system. The frame member may also assist in maintaining the rigidity and/or strength of the synthetic metal system, may enable the frame member to be in at least partial contact with the environment external to the synthetic metal system, may provide the synthetic metal system with desired properties, or the like.

In an alternative embodiment of the invention, the frame member may be substantially different in size, shape, and configuration to the synthetic metal system. In this instance, it is envisaged that the frame member may not affect the rigidity or strength of the synthetic metal system, may be at least partially insulated from contact with the environment external to the synthetic metal system, or the like. In some embodiments of the invention, the frame member may improve the strength of the synthetic metal system. In addition, the frame member may increase the strength of a component with which the synthetic metal system is being used. Further, the frame member may be used to replace an existing component of a system or device by providing improved strength and/or durability over an existing component. Alternatively, the frame member may provide equivalent strength to an existing component but may result in reduced weight and/or material requirement in comparison to an existing component.

The frame member may be of any suitable configuration. For instance, the frame member may be non-porous, may comprise a cellular structure, may be porous, or any suitable combination thereof. Preferably, the frame member may comprise a cellular structure (such as, but not limited to, a honeycomb structure), wherein the cellular structure comprises a plurality of openings. In some embodiments of the invention, the plurality of openings in the cellular structure may comprise pores or voids in a material (such as a foamed material, a naturally porous material such as a cementitious material, an open cell structured foam or a closed cell structured foam, or the like). In some embodiments of the invention, the openings may be connected to each other and form an interconnected network or may not be interconnected. In some embodiments of the invention, the plurality of openings defines a cavity within and/or on a surface of the frame member. However, it will be understood that the type and location of the cavity may vary depending on a number of factors such as the object to be inserted into and/or through the frame member and the application of the synthetic metal system. In use, it is envisaged that the plurality of openings enables the substantially unimpeded flow of the matrix material throughout the frame member.

The frame member may comprise one or more regions, wherein the regions exhibit different characteristics. For instance, the different regions may have different stiffness values, electrical conductivity, resistance to abrasion, magnetic properties, heat sensitivity, thermal conductivity, corrosion and/or chemical resistance, metallurgy, weight, density, radioactivity, strength to weight ratio, surface area to volume ratio, impact resistance, vibration dampening, or the like. It is envisaged that providing the frame member with regions exhibiting different characteristics may provide the synthetic metal system with regions having different properties. In some embodiments of the invention, the frame member may comprise one or more regions which are substantially non-porous. In use, it is envisaged that the one or more regions of the frame member may be formed by connecting one or more portions of a frame member to each other, by three-dimensional printing a pattern of multiple materials during fabrication of the frame member, by depositing, plating or coating a frame member with one or more different materials, by customising the machining parameters or printer settings for each region to provide the regions with different properties, or any suitable combination thereof.

In a preferred embodiment of the invention, the plurality of openings in the cellular structure may be defined by a plurality of frame elements (e.g. strips, bars, girders, beams, or the like) which contact, abut, cross and/or overlap one another to form the frame member. The frame elements may be straight, curved, angled or any suitable combination thereof. In this embodiment, the frame elements may be provided in a regular pattern, or an irregular pattern. In some embodiments of the invention, the frame elements may be provided in a Voronoi pattern. However, it will be understood that any suitable pattern may be used providing the pattern of frame elements facilitates the penetration of the matrix material into the openings in the frame member whilst providing the frame member with the desired physical properties, such as strength to weight ratio, surface area to volume ratio, impact resistance, shock absorption, vibration dampening, or the like.

The openings defined by the frame elements may be of any suitable size, shape or configuration. Preferably, the frame member may be a lattice structure comprising a micro-architecture including a network of nodes and frame elements. Any suitable lattice structure may be used. Preferably, however, the lattice structure may be of a sufficient size, shape or configuration to provide the frame member with the desired physical properties, such as strength to weight ratio, surface area to volume ratio, impact resistance, shock absorption, vibration dampening, or the like, whilst providing a pattern of frame elements which facilitates the penetration of the matrix material into the openings in the frame member. In use, it is envisaged that the lattice structure may assist in the providing a conduit for the transfer of electrons and formation of an electrical circuit.

In an embodiment of the invention, one or more frame elements may be at least partially hollow. In this instance, it is envisaged that providing hollow frame elements may reduce the density of the frame member, may provide a conduit for the transfer of a fluid material (such as a refrigerant, a coolant a gas, a powder, a liquid, a molten material, or the like) through the frame elements, may be at least partially with a filler material (such as a settable material, a powder, a molten material, an expandable material, or the like), may provide a conduit for an elongate material (such as fibre optic strands, fibres fabricated from a polymeric or metallic material, or the like) or any suitable combination thereof. In this instance, it is envisaged that by providing the frame member with hollow frame elements, it may enable the physical properties of the frame member to be altered. For instance, a frame member comprising hollow frame elements with a copper filler material may be conductive. For instance, a frame member with hollow frame elements comprising a refrigerant or coolant may assist in maintaining the operating temperature of the synthetic metal system. The hollow frame elements may be temporarily or permanently filled. For instance, the hollow frame elements may be filled with a refrigerant or coolant during operation of the synthetic metal system and emptied during storage of the synthetic metal system. For instance, the hollow frame elements may be permanently filled with a settable material or may be reversibly filled. For instance, the hollow frame elements may be filled with a material during fabrication of the synthetic metal system which is then removed (such as by dissolving, solubilising, melting, or the like) after fabrication.

In some embodiments of the invention, the frame member may comprise a multi-material structure. The frame member may comprise any suitable multi-material structure, however, it will be understood that the type of structure may vary depending on a number of factors such as the desired properties of the synthetic metal system, the type of materials used and the type of fabrication process. In some embodiments of the invention, the frame member may be provided with a plating or a coating. In this instance, it will be understood that an exposed surface of the frame member, such as an opening, a frame element, or the like may be plated or coated. For instance, the frame member may be provided with a copper plating to provide the frame member with improved thermal or electrical conductivity, may be provided with a nickel plating to provide the frame member with improved resistance to corrosion, may be coated with hydrophilic or hydrophobic polymers to modify the surface properties of the frame member, or the like. In other embodiments of the invention, two or more layers may be formed during the fabrication process. For instance, two or more materials may be deposited during three-dimensional printing, may be co-extruded, or the like. In some embodiments of the invention the two or more materials may be the same type of material or different types of materials.

In some embodiments of the invention, the frame member may be relatively rigid. The term “rigid” when referring to the frame member refers to a structure showing a limited degree of flexibility, more particularly, the rigidity ensures that the structure forms and retains a predefined shape in a three-dimensional space prior to, during and after use and that this overall shape is mechanically and/or physically resistant to pressure applied thereto. In a preferred embodiment of the invention the frame member may be a relatively rigid cellular structure.

In an alternative embodiment of the invention, the frame member may be relatively flexible or non-rigid. Preferably, the frame member may be a relatively flexible or non-rigid cellular structure. It will be understood that a relatively flexible or non-rigid frame member may at least partially deform in overall shape in response to pressure applied to the frame member. In a preferred embodiment of the invention, the frame member may be resiliently deformable, wherein the frame member recovers to substantially its original state in response to release of the pressure applied to the frame member. Despite the overall rigidity of the shape of the envisaged cellular structure, the specific stiffness and density of the structure may be determined by the configuration of the cellular structure (such as, but not limited to, the pattern and density of the openings, the size and geometries of the openings, the size, shape and configuration of the frame elements) and/or material of the cellular structure. However, it will be understood that the properties of the cellular structure may vary depending on the desired physical properties of the frame member.

In some embodiments of the invention, the frame member may comprise a nonuniform cellular structure wherein the size, shape and configuration of the openings and/or frame elements may vary, or a uniform cellular structure. In some embodiments of the invention, the frame member may comprise a cellular structure wherein the size, shape and configuration of the openings and/or frame elements may be randomly distributed, or may be engineered. However, it will be understood that the size, shape and configuration of the cellular structure of the frame member may vary depending on the method of fabrication and the desired physical properties of the synthetic metal system.

The frame member may be fabricated using any suitable technique. For instance, the frame member may be fabricated using liquid metal foaming, dealloying, combustion, electrodeposition, sintering of metal powders at elevated temperature, template-based fabrication (such as carbon templating, polymer directed self-assembly, polymer melt mixing, polymer foaming), additive manufacturing or three-dimensional printing (such as stereolithography, selective layer melting, fused deposition modelling, powder bed fusion, direct energy deposition, direct metal deposition, laminated object manufacture, microscale stereolithography, nanoscale stereolithography, multiphoton lithography, femtosecond projection two photon lithography, microlaser sintering, or the like), metal machining (such as metal stamping, metal forming, laser cut, metal pressing, metal broaching, metal shaving, or the like) or any suitable combination thereof. In a preferred embodiment of the invention, the frame member may be fabricated using a three-dimensional printing method. In a preferred embodiment of the invention, the frame member may be manufactured using Selective Laser Sintering (SLS), Direct Metal Laser Sintering (DSLS) or CNC Machining.

The frame member may be of unitary structure or may be fabricated from two or more portions that are configured for connection to one another and/or are retained in place relative to one another within the synthetic metal system by the matrix material. The two or more portions may be placed in abutment with one another, at least partially overlapping one another, or spaced apart from one another. In some embodiments of the invention, the two or more portions may be intertwined. In some embodiments of the invention, one or more inner portions of the frame member may be at least partially received within an outer portion of the frame member. In other embodiments of the invention, one or more inner portions of the frame member may be substantially received within an outer portion of the frame member. In this instance, the one or more inner portions of the frame member may be received within a cavity in the outer portion of the frame member. In use, it is envisaged that a three-dimensional printing method may provide the ability to fabricate a frame member comprising two or more portions, wherein one or more inner portions of the frame member may be located within a cavity in the outer portion of the frame member or wherein the two or more portions are intertwined or form a complex shape or configuration.

In some embodiments of the invention, the two or more portions of the frame member may be the same size, shape or configuration or may be a different size, shape or configuration. In some embodiments of the invention, the two or more portions may be of the same type of material or may be of different types. It is envisaged that providing the frame member with two or more portions having a different size, shape or configuration or fabricated from different types of material may provide the synthetic metal system with regions having different properties. For instance, the two or more portions may have different stiffness values, electrical conductivity, resistance to abrasion, magnetic properties, heat sensitivity, thermal conductivity, corrosion and/or chemical resistance, metallurgy, weight, density, radioactivity, strength to weight ratio, surface area to volume ratio, impact resistance, vibration dampening, or the like. For instance, the two or more portions may be used as a conduit for different materials (such as coolants, hot liquids, gases, or the like). In this embodiment, it is envisaged that the frame member may act as a heat exchange device.

The frame member may be fabricated from any suitable material. For instance, the frame member may be fabricated from a polymeric material, a metallic material, a ceramic material, a composite material, or any suitable combination thereof. In a preferred embodiment of the invention, the frame member may be fabricated from a conductive material. Preferably, the frame member may be fabricated from an electrically conductive material or a thermally conductive material. In an embodiment of the invention, the frame member may be fabricated from a biocompatible material. In some embodiments of the invention, the frame member may be fabricated from two or more materials. In some embodiments of the invention, the frame member may be fabricated from a composite material. In some embodiments of the invention, the frame member may be fabricated from a fibre reinforced material. Any suitable fibre may be used to reinforce the material, such as aramid fibre, glass fibre, carbon fibre, or the like. The material being reinforced may be of any suitable type, such as a polymeric material, a carbon material, a metallic material, or any suitable combination thereof. For instance, the frame member may be fabricated from a carbon fibre composite such as a carbon fibre reinforced polymer, a carbon fibre reinforced carbon, or the like.

In a preferred embodiment of the invention, the frame member may be fabricated from a metallic material. In some embodiments of the invention, the frame member may be fabricated from a thermosetting thermoplastic material to form a lattice structure. In this instance, a metallic material may be coated on the thermoplastic material lattice structure, may be used to at least partially penetrate one or more openings in the thermoplastic material lattice structure, or the like.

Any suitable metallic material may be used, although it will be understood that metallic materials having one or more desirable physical properties (such as being relatively lightweight, relatively durable, relatively conductive, relatively magnetic, relatively resiliently deformable and so on) may be preferred. Thus, in specific embodiments of the invention, the frame member may be fabricated from steel (including mild steel, stainless steel or the like), titanium or alloys thereof, zirconium or alloys thereof, aluminium or alloys thereof, copper or alloys thereof, or the like. In an embodiment of the invention, the frame member may comprise a non-metallic cellular structure coated with a metallic material. However, the exact composition of the metallic material may vary depending on the relevant reference standard, the desired physical properties and the technology or field in which the synthetic metal system will be used.

For instance, where the synthetic metal system may be used to fabricate a sacrificial anode, it is envisaged that the frame member may be fabricated from zinc, aluminium, magnesium, or an alloy thereof. For instance, the frame member may be at least partially constructed from a material which dissolves or melts in specific environmental conditions or after a period of time.

For instance, where the synthetic metal system may be used to fabricate a component for electrical circuitry applications or other electronic systems, it is envisaged that the frame member may be fabricated from copper or an alloy thereof. For instance, where the synthetic metal system may be used to fabricate a two-pin plug or a three-pin plug for an electric appliance, it is envisaged that the conductive pins (such as earth, positive terminal, negative terminal, hot wire, neutral wire, or the like) may be a frame member fabricated from copper, wherein the matrix material is an insulator.

For instance, where the synthetic metal system may be used to fabricate a component for marine use, it is envisaged that the frame member may be fabricated from stainless steel. For instance, where the synthetic metal system may be used to fabricate a component for aeronautical use, it is envisaged that the frame member may be fabricated from aluminium or an alloy thereof. For instance, where the synthetic metal system may be used to fabricate a component for use in space or other thermo-vacuum cyclic environments, it is envisaged that the frame member may be fabricated from titanium or an alloy thereof.

For instance, where the synthetic metal system may be used to fabricate a component, such as a prosthetics, implants, surgical devices or instruments, or the like, for use in medical or dental applications, it is envisaged that the frame member may be fabricated from titanium or an alloy thereof. In use, it is envisaged that a component fabricated from a synthetic metal system may better mimic the characteristics and mechanics of the body part being replaced, may be more readily custom-fit for each patient, may be more efficiently and economically designed and fabricated, may have improved initial fixation, may provide improved bone in-growth potential, or the like. In use, surgical devices or instruments fabricated from a synthetic metal system may have decreased development time and decreased time-to-market.

For instance, the synthetic metal system may be used to fabricate a light emitting system (such as a torch, a light bulb, a light emitting diode, a high intensity discharge lamp, a compact fluorescent lamp, a halogen lamp, a metal halide lamp, or the like). For instance, where the synthetic metal system may be used for liquid heat exchange, the frame member may comprise one or more inner portions substantially received within an outer portion of the frame member, wherein some portions of the frame member may be a conduit for a cool fluid and other portions of the frame member may be a conduit for a heated fluid. For instance, where the synthetic metal system may be used as a vibration damping or shock absorbing element, the frame member may be a resiliently deformable member substantially encased within a non-rigid matrix material. In this instance, it is envisaged the frame member may be fabricated from a resiliently deformable material and/or the shape and configuration of the frame member may provide the frame member with these properties.

For instance, where the synthetic metal system may be used to fabricate a projectile, it is envisaged that the frame member may be fabricated from lead, copper, or alloys thereof and the matrix material may be fabricated from a relatively non-reactive polymer such as liquid crystal polymer or other injection moulded polymer material such as Nylon or similar. Preferably, the frame member may be fabricated from a metal other than lead. Preferably the frame member may be fabricated from a material that is relatively harmless to the environment. For instance, the frame member may be fabricated from aluminium or an aluminium alloy, stainless steel, titanium or titanium alloy, or the like. The plurality of openings in the frame member may be in any suitable pattern. In some embodiments of the invention, the plurality of openings in the frame member may be formed by a Voronoi pattern. In use, it is envisaged that a synthetic metal system projectile may comprise a matrix material such as a self-lubricating polymer and/or abrasion resistant polymer. Preferably, the synthetic metal system projectile may comprise a matrix material developed for high impact forces or ballistics applications.

In an alternative embodiment of the invention, the frame member of the synthetic metal system projectile comprises two or more portions, wherein the frame member portions may be configured for connection to one another or may be retained in place relative to one another within the synthetic metal system by the matrix material. The frame member portions may be of the same configuration or may be of different configurations. For instance, a first frame member portion may form the upper portion of the frame member and a second frame member portion may form the lower portion of the frame member, wherein the first frame member portion may be fabricated with a tip and the second frame member portion may be fabricated with a base. For instance, the frame member may comprise two or more frame member portions having a substantially similar plate-like configuration, wherein a region of each of the frame member portions may be configured for connection to one another. In this instance, it is envisaged that the cross section of the assembled frame member may be substantially symmetrical.

In some embodiments, a marker such as micro-identifying chemicals, micro RFID tags, printed plastic, or the like may be added to the matrix material of the synthetic metal system projectile. In this instance, the marker may facilitate ballistic forensic tracing, inventory management, or the like. In some embodiments of the invention, the synthetic metal system projectile may have the same size, shape, or configuration as a conventional projectile.

In some embodiments of the invention, the base of the synthetic metal system projectile may be integral with the frame member. In this instance, it is envisaged that the base may be configured such that an explosive force may be focussed on a desired portion of the projectile. For instance, providing the synthetic metal system projectile with a concave base may focus the explosive force on the bottom centre zone of the projectile. It is envisaged that a projectile fabricated from a synthetic metal system may have reduced felt recoil, increased velocity, and increased kinetic energy relative to a conventional nickel-plated bullet or other solid metal projectiles. As a result, projectiles fabricated from a synthetic metal system may have reduced over-penetration and improved stopping power.

In some embodiments of the invention, the frame member may be fabricated from a metallic material having a more negative reduction potential or more positive electrochemical potential than the structure with which the synthetic metal system is to be associated or connected to. In this way, the frame member will corrode in preference to the structure.

The synthetic metal system comprises a matrix material. The matrix material may be configured to at least partially penetrate the openings in the frame member such that the frame member is at least partially encased within the matrix material. Any suitable portion of the frame member may be encased within the matrix material. For instance, the matrix material may at least partially coat an outer surface of the frame member, may substantially encase the frame member, may at least partially coat an opening in the frame member, may substantially fill an opening in the frame member, may at least partially fill an interconnected network of openings in the frame member, may substantially fill an interconnected network of openings in the frame member, may substantially fill all interconnected networks of openings in the frame member, or any suitable combination thereof. Preferably, however, the matrix material may bond to the frame member so that the frame member and the matrix material may not separate from one another. Preferably, the matrix material may be configured to substantially penetrate openings in the frame member such that the frame member may be substantially encased within the matrix material.

In some embodiments of the invention, the matrix material may be relatively rigid, or may be relatively flexible or non-rigid when set. It will be understood that the rigidity of the matrix material may vary depending on a number of factors, such as the type of matrix material used, the size, shape or configuration of the frame member relative to the size, shape or configuration of the synthetic metal system and the desired properties of the synthetic metal system. In a most preferred embodiment of the invention, both the matrix material and the frame member may be resiliently deformable. It is envisaged that the matrix material and the frame member may deform with one another, such as upon impact or when a force is applied. In this way, the integrity of the synthetic metal system may be maintained. For instance, if the frame member was significantly less resiliently deformable than the matrix material, it is possible that damage to, or breakage of, the matrix material may occur when a force is applied. Not only would this damage the structural integrity of the synthetic metal system, but it may also increase the susceptibility of the system to corrosion and/or decrease the conductivity of the system.

In some embodiments of the invention, the matrix material may be of varying thickness across the outer surface of the frame member or may be relatively uniform throughout. It will be understood that the thickness of the matrix material may vary depending on a number of factors, such as the size, shape or configuration of the frame member relative to the size, shape or configuration of the synthetic metal system and the desired properties of the synthetic metal system. For instance, the matrix material may form a relatively thin layer across an outer surface of the frame member such that the outer surface of the frame member is proximate the outer surface of the matrix material. In use, it is envisaged that a synthetic metal system comprising a frame member having a relatively thin coating of matrix material may improve conductivity of the synthetic metal system or may increase the rate of exposure of the frame member by abrasion. For instance, the matrix material may form a relatively thick layer across an outer surface of the frame member such that the outer surface of the frame member may not be exposed through the outer surface of the matrix material by abrasion. In some instances, the synthetic metal system may comprise one or more regions, wherein the regions comprise different thicknesses of the matrix material. It is envisaged that providing the synthetic metal system with regions exhibiting different characteristics may provide the synthetic metal system with regions having different properties.

In an embodiment of the invention, at least a portion of the frame member may be in at least partial contact with the environment external to the synthetic metal system. For instance, the frame member may be at least partially exposed through an outer surface of the matrix material, that is, at least a portion of the frame member may be external relative to the outer surface of the matrix material. In an embodiment of the invention, the at least a portion of the frame member exposed to the environment may act as a conduit of electrons. In this instance, the conduct of electrons through the frame member may produce a current sufficient to charge an external device or may provide cathodic protection to a surface that the synthetic metal system may be in contact with, that is the frame member may act as a sacrificial anode. In an embodiment of the invention, the at least a portion of the frame member exposed to the environment may assist in heat transfer away the synthetic metal system by increasing convection. In this instance, the portion of the frame member exposed to the environment may comprise a projection (such as a fin, a flute, or the like) extending outwardly from the frame member.

In an embodiment of the invention, the at least a portion of the frame member exposed to the environment may be adapted for connection to any suitable object (such as an adjacent synthetic metal system, a component of a device, a device, a structure, or the like). For instance, the at least a portion of the frame member may be connected to an object by frictional engagement, by complementary connection portions (such as screw-threaded sections, splined sections, locking sections or the like, or any suitable combination thereof), by one or more mechanical fasteners, by an adhesive, by a heat or chemical treatment, or by welding using any suitable technique.

In an embodiment of the invention, the frame member is substantially encased within the matrix material. In this instance, the frame member may not be exposed through an outer surface of the matrix material, that is, substantially all of the frame member may be internal relative to the outer surface of the matrix material.

The matrix material may comprise any suitable organic or inorganic material. For instance, an inorganic matrix material may comprise a metallic material, a ceramic material, a carbon-carbon composite, a glass, a silicate, a silicon or boron nitride, or any suitable combination thereof. For instance, an organic matrix material may comprise a polymeric material, a rubber or silicone material, a gel forming material, or any suitable combination thereof. Preferably, however, the matrix material exhibits abrasion-resistance, impact-resistance, tear and kink-resistance, chemical-resistance, oil-resistance and grease-resistance and excellent resistance to hydrolysis and microbiological attack. Preferably, the matrix material may be suitable for processing by injection moulding and/or extrusion. In use, it is envisaged that the heat of injection moulding and/or extrusion may polymerise and/or melt the matrix material, wherein the liquid form of the matrix material may be sufficient to flow and cover the frame member. In an embodiment of the invention, the matrix material may be fabricated from a biocompatible material or other suitable medical grade material. However, it will be understood that the type and composition of the matrix material may vary depending on a number of factors, such as the application of the synthetic metal system and the type of frame member.

The matrix material may be of any suitable form. For instance, the matrix material may be in the form of grains, powders, pellets, or the like. However, it will be understood that the form of the matrix material may vary depending on a number of factors including the type of injection unit, the type of matrix material and the desired properties of the synthetic metal system.

In a preferred embodiment of the invention, the matrix material may comprise a polymeric material or any suitable blend thereof. For instance, the polymeric material may comprise one or more monomers, one or more polymers, one or more copolymers, or any suitable combination thereof. Preferably, the matrix material may comprise one or more thermoplastic, thermosetting and/or elastomeric polymeric materials. Any suitable polymeric material may be used. In a preferred embodiment of the invention, the matrix material may be fabricated from a non-hygroscopic polymer material.

However, it will be understood that the type of polymeric material used in the matrix material may vary depending on the use of the synthetic metal system. For instance, a matrix material comprising a polyamide (such as Nylon) would not be suitable to fabricate a component for use in space or other thermo-vacuum cyclic environments as nylons depolymerise at relatively low temperatures in vacuum, however a matrix material comprising acetal or polycarbonate may be suitable for this application. For instance, where the synthetic metal system may be used to fabricate a component for an internal combustion engine, it is envisaged that the matrix material may comprise a high temperature polymer. For instance, where the synthetic metal system may be used to fabricate a projectile, it is envisaged that the matrix material may comprise a liquid crystal polymer which has high mechanical strength at high temperatures and is substantially resistant to chemicals, weathering, radiating and/or burning.

The matrix material may comprise, for instance, polyethylene, polypropylene, polystyrene, polyvinyl chloride, polyether sulphones, acrylonitrile butadiene styrene, polyether polyols, polyester polyols, polyacetal or polyoxymethylene (acetal, POM), polycarbonates, elastomers, thermoplastic elastomers, thermoplastic polyester, thermoplastic polyurethane, polyketones (such as but not limited to polyaryletherketone (PAEK), polyetheretherketone (PEEK), polyetherketoneketone (PEKK) or the like), polyphenylene sulfide (PPS), polyamides, polyphthalamide (PPA, performance Nylon), fluoropolymers (such as, but not limited to, polytetrafluoroethylene (PTFE), fluorinated ethylene propylene (FEP), polyvinylidene fluoride or polyvinylidene difluoride (PVDF), polychlorotrifluoroethylene (PCTFE), perfluoroalkoxy alkane (PFA), or the like), high density polyethylene (HDPE), ultra-high molecular weight polyethylene (UHMWPE), epoxy resins, phenolic resins, and the like, their monomers thereof, or any suitable combination thereof. In an embodiment of the invention, the matrix material may comprise a polyketone or a fluoropolymer.

The matrix material may be processed by injection moulding. In an embodiment of the invention, a multi-phase chemo-synthetic copolymer thermoplastic polyurethane matrix is used. The preferred polymer is formed from the inter-reaction of three components: polyols (long-chain diols); diisocyanates; and short-chain diols. The polyols and the short-chain diols react with the diisocyanates through polyaddition to form linear polyurethane. Flexible segments are created by the reaction of the polyol with the diisocyanate. The combination of diisocyanate with short-chain diol produces the rigid component (rigid segment). The properties of the particular polymer used will depend on the nature of the raw materials, the reaction conditions, and the ratio of the starting materials to achieve the properties of the polymer desired. The polyols used have a significant influence on certain properties of the resultant thermoplastic polyurethane. Either polyester-based polyols or polyether-based polyols may be used in the production of the matrix material. For instance, using polyester polyols provides highest mechanical properties, highest heat resistance, and highest resistance to mineral oils, whereas using polyether polyols provides highest hydrolysis resistance, best low-temperature flexibility, and resistance to microbiological degradation.

In some embodiments of the invention, the matrix material may contain additives to facilitate production and processability. Further additives can also be included to modify specific properties. Such additives include mould release agents, flame retardants, lubricants, UV-stabilizers, plasticizers, gel forming materials, or the like. Fillers, such as aramid fibre, glass fibre, carbon fibre, and mineral filler in flake or power form may be used to modify the tensile strength and stiffness of a product, regulate shrinkage, improve wear resistance, improve processability, and the like. In an embodiment of the invention, the synthetic metal system comprises a non-fibre reinforced matrix material. In an embodiment of the invention, the synthetic metal system comprises a fibre reinforced matrix material.

It is envisaged that, when the frame member is substantially surrounded by the matrix material to form the synthetic metal system, the frame member provides improved mechanical properties (such as strength, rigidity, insulation, impact resistance and the like, or any suitable combination thereof) to the synthetic metal system. In particular, it is envisaged that the mechanical properties of the frame member may be greater than the mechanical properties of the matrix material. Thus, it is envisaged that the frame member may effectively act as an endoskeleton about which the relatively soft matrix material is applied. In some embodiments of the invention, the frame member and the matrix material may have similar mechanical properties. Thus, it is envisaged that the frame member may effectively act as a relatively flexible endoskeleton about which a relatively soft matrix material is applied. In yet other embodiments of the invention, the mechanical properties of the matrix material may be greater than the mechanical properties of the frame member. Thus, it is envisaged that the frame member does not effectively contribute to the mechanical properties of the synthetic metal system.

In an embodiment of the invention, the synthetic metal system may be associated with a well bore. For instance, the synthetic metal system may be associated with a well casing, a portion of a pipeline, a bottom hole assembly (such as, but not limited to, drilling equipment, an artificial lift pump system, a gas lift system, a submersible pump, a hydraulic pump system, a jet pump system, a slurry pump, or the like), a portion of a drill string, a portion of a production tubing string, a portion of a sucker rod string, or the like. In an embodiment of the invention, the synthetic metal system may be associated with a bottom hole assembly. In a preferred embodiment of the invention, the synthetic metal system may be associated with a pump system, such as, but not limited to, a rod pump, a hydraulic pump, an electric submersible pump (ESP), a gas lift, a plunger lift, a progressive cavity pump, of the like. The synthetic metal system may be associated with any suitable component of the pump system. For instance, the system synthetic metal system may be associated with a pump, a motor, a cable, a valve, a portion of a rod string, a cable, a gas lift mandrel, a plunger, a valve, an injection system, a venturi system, a mixer, a roller or wheel, or any suitable combination thereof. In this instance, it is envisaged that the synthetic metal system may provide the bottom hole assembly with a property of, or an improved property of, electrical conductivity, resistance to abrasion, magnetic properties, heat sensitivity, thermal conductivity, corrosion and/or chemical resistance, radioactivity, strength to weight ratio, surface area to volume ratio, impact resistance, vibration dampening, or the like.

In an embodiment of the invention, the synthetic metal system may be associated with a sucker rod pump system of a bottom hole assembly. The synthetic metal system may be associated with any suitable component of the sucker rod pump system. For instance, the synthetic metal system may be associated with a casing, a production tubing, a rod string, or the like, or any suitable combination thereof. In an embodiment of the invention, the synthetic metal system may be associated with a rod string of the sucker rod pump system. It will be understood that the term “rod string” is intended to refer to certain components of the sucker rod pump system, such as, but not limited to a sucker rod, a pony rod, a coupling, a rod guide, a centralising assembly, and so on. The synthetic metal system may be associated with any suitable portion of the rod string. In an embodiment of the invention, the synthetic metal system may be associated with a sucker rod and/or a pony rod. In a preferred embodiment of the invention, the synthetic metal system may be associated with a centralising assembly of the rod string. In a preferred embodiment of the invention, the synthetic metal system may be associated with a rod guide of the rod string. Preferably, however, the synthetic metal system may be associated with a portion of the sucker rod pump system such that the synthetic metal system provides cathodic protection to the sucker rod pump system and/or a portion of the well bore and may be relatively convenient to monitor and/or replace.

Preferably, the synthetic metal system may be adapted for connection to, or retention in, the casing, the production tubing, and/or the rod string of a sucker rod pump system. In an embodiment of the invention, the synthetic metal system may be adapted for removable connection to, or retention in, the casing, the production tubing, and/or the rod string of a sucker rod pump system. In an embodiment of the invention, the synthetic metal system may be adapted for permanent connection to, or retention in, the casing, the production tubing, and/or the rod string of a sucker rod pump system. In this instance, it is envisaged that a portion of the casing, the production tubing, and/or the rod string comprising the synthetic metal system may be adapted for removable connection such that a worn or damaged synthetic metal system may be replaceable.

The synthetic metal system may be connected to, or retained in, the casing, the production tubing, and/or the rod string of the sucker rod pump system by any suitable means. For instance, the synthetic metal system may be removably connected to, or retained in, the casing, the production tubing, and/or the rod string by frictional engagement, or by providing complementary screw-threaded portions, press fittings, clip-on fittings, snap-fit features, male-female connectors, or combinations thereof. For instance, the synthetic metal system may be connected to the casing, the production tubing, and/or the rod string using one or more mechanical fasteners (screws, bolts, rivets, pins, or the like), an adhesive, a heat or chemical treatment, or welded using any suitable technique. For instance, the synthetic metal system may be overmoulded to at least a portion of the casing, the production tubing, and/or the rod string such that the synthetic metal system may be integrally moulded to at least a portion of the rod string.

The synthetic metal system may replace one or more components or parts of the casing, the production tubing, and/or the rod string of a sucker rod pump system. Preferably, the synthetic metal system may replace and function as the one or more components of the casing, the production tubing, and/or the rod string. It will be understood that the term “function” is intended to refer to the purpose or operation of that component within the casing, the production tubing, and/or the rod string, that is, the synthetic metal system will effectively function as the component it is replacing in addition to providing one or more properties of a synthetic metal. The synthetic metal system may replace and function as any suitable component of the casing, the production tubing, and/or the rod string. For instance, suitable components may include a sucker rod, a pony rod, a coupling, a rod guide, a centralising assembly, and the like. Preferably, however, the synthetic metal system may replace a component of the casing, the production tubing, and/or the rod string such that the synthetic metal system provides cathodic protection to the casing, the production tubing, and/or rod string during well pumping. In a preferred embodiment of the invention, a rod string of a sucker rod pump system comprises one or more centralising assemblies and/or one or more rod guides, wherein the centralising assembly and/or the rod guide is replaced by a synthetic metal system and wherein the synthetic metal system provides cathodic protection to the rod string during well pumping.

In an embodiment of the invention, the synthetic metal system may be associated with an electric submersible pump system. The synthetic metal system may be associated with any suitable component of the electric submersible pump system. For instance, the synthetic metal system may be associated with a casing, a production tubing, motor protectors, or the like, or any suitable combination thereof. Preferably, however, the synthetic metal system may be associated with a portion of the electric submersible pump system such that the synthetic metal system provides cathodic protection to the electric submersible pump system and/or a portion of the well bore and may be relatively convenient to monitor and/or replace.

Preferably, the synthetic metal system may be adapted for connection to, or retention in, the casing, the production tubing, and/or the motor protectors of an electric submersible pump system. In an embodiment of the invention, the synthetic metal system may be adapted for removable connection to, or retention in, the casing, the production tubing, and/or the motor protectors of an electric submersible pump system. In an embodiment of the invention, the synthetic metal system may be adapted for permanent connection to, or retention in, the casing, the production tubing, and/or the motor protectors of an electric submersible pump system. In this instance, it is envisaged that a portion of the casing, the production tubing, and/or the rod string comprising the synthetic metal system may be adapted for removable connection such that a worn or damaged synthetic metal system may be replaceable.

The synthetic metal system may be connected to, or retained in, the casing, the production tubing, and/or the motor protectors of an electric submersible pump system. For instance, the synthetic metal system may be removably connected by frictional engagement, by complementary male female connection portions, by one or more mechanical fasteners, by an adhesive, by a heat or chemical treatment, by welding, by overmoulding, or the like.

The synthetic metal system may replace one or more components or parts of the casing, the production tubing, and/or the motor protectors of an electric submersible pump system. Preferably, the synthetic metal system may replace and function as the one or more components of the casing, the production tubing, and/or the motor protectors. It will be understood that the term “function” is intended to refer to the purpose or operation of that component within the casing, the production tubing, and/or the motor protectors, that is, the synthetic metal system will effectively function as the component it is replacing in addition to providing one or more properties of a synthetic metal. The synthetic metal system may replace and function as any suitable component of the casing, the production tubing, and/or the motor protectors. Preferably, however, the synthetic metal system may replace a component of the casing, the production tubing, and/or the motor protectors such that the synthetic metal system provides cathodic protection to the casing, the production tubing, and/or the motor protectors during well pumping.

The synthetic metal system may replace one or more components or parts of the sucker rod pump system, the electric submersible pump system, or the like. The components or parts of the sucker rod pump system, the electric submersible pump system, or the like, being replaced may be of any suitable type, for instance, the components may be an original equipment manufacturer part, a genuine part, an aftermarket part, or the like. Alternatively, the components or parts of the sucker rod pump system, the electric submersible pump system, or the like, being replaced may be a modified part, a remanufactured part, a refurbished part, or the like. However, it will be understood, that the type of part may vary depending on a number of factors, such as whether the sucker rod pump system, an electric submersible pump system, or the like is new or second-hand, user preferences and the type of part.

In an embodiment of the invention, the synthetic metal system may be used to fabricate a portion of a ball. The synthetic metal system may be used in any suitable ball. For instance, the ball may be a golf ball, a cricket ball, a baseball, a softball, a tee-ball, a hockey ball, or the like. The synthetic metal system may be used to fabricate any suitable portion of the ball. For instance, the synthetic metal system may be used to fabricate a core, a mantle layer or a cover of a ball.

Preferably, the synthetic metal system may be used to fabricate a core and/or one or more mantle layers of a ball, wherein the core and/or the mantle layer may be resiliently deformable. The term “resiliently deformable” when referring to the core or the mantle layer refers to a structure showing the ability to at least partially deform in overall shape in response to pressure applied to the structure and which recovers to substantially its original state in response to the release of the pressure applied to the structure. In use, it is envisaged that a resiliently deformable core and/or mantle layer assists in the transfer of energy from an object (such as, but not limited to a hand, a foot, a racquet, a bat, a club, or the like) to the ball. In use, it is envisaged that a ball comprising a synthetic metal system may have a shape memory, enabling the ball to deform and then spring back into its original shape following contact with an object. In use, it is envisaged that a ball comprising a synthetic metal system may be designed to produce a desired frequency vibration or not produce an undesired frequency vibration, following contact with an object. In use, it is envisaged that a ball comprising a synthetic metal system cover may reduce the chances of the ball cover splitting due to impact and especially impact by a bat, a club, or other hard surface.

The synthetic metal system may be used to fabricate any suitable portion of the core and/or the mantle layer of a ball. For instance, the synthetic metal system may be used to fabricate substantially the entire core, one or more layers of the core, one or more hemispheres of the core, one or more segments of the core (such as, but not limited to, spherical areas, crescents, wedges, skullcaps, or the like), or any suitable combination thereof. For instance, the synthetic metal system may be used to fabricate substantially an entire mantle layer, one or more layers of a mantle layer, one or more hemispheres of a mantle layer, one or more segments of a mantle layer (such as, but not limited to, spherical areas, crescents, wedges, skullcaps, or the like), or any suitable combination thereof. In some embodiments of the invention, the ball may comprise two or more synthetic metal systems. The two or more synthetic metal systems may be the same type of synthetic metal system or may be of different types. For instance, the ball may comprise a core fabricated from a first synthetic metal system and a mantle layer fabricated from the same or a different synthetic metal system. For instance, the ball may comprise a first mantle layer fabricated from a first synthetic metal system and a second mantle layer fabricated the same or a different synthetic metal system.

However, it will be understood that the portion of the core and/or mantle layer of the ball fabricated from the synthetic metal system may vary depending on the type of ball and the desired characteristics of the ball. In use, it is envisaged that using a synthetic metal system to fabricate one or more portions of the core and/or the mantle layer of the ball or using different synthetic metal systems may provide the ball with one or more regions exhibiting different characteristics. For instance, the different regions may have different stiffness values, impact resistance, vibration dampening, resistance to deformation, strength to weight ratio, surface area to volume ratio, density, or the like.

Preferably, the synthetic metal system may replace and function as the portion of a core and/or a mantle layer of the ball. It will be understood that the term “function” is intended to refer to the purpose or operation of that portion of the core and/or the mantle layer of the ball, that is, the synthetic metal system will effectively function as the portion of the core and/or the mantle layer of the ball it is replacing in addition to providing one or more properties of a synthetic metal. The synthetic metal system may replace and function as any suitable portion of the core of the ball and/or the mantle layer. In use, it is envisaged that providing a ball with a core and/or a mantle layer fabricated from a synthetic metal system may reduce the inter-variability of the distribution of density or specific gravity between balls and may improve the consistency of the distribution of density or specific gravity within a ball.

In some embodiments of the invention, the synthetic metal system may provide additional functionality to the ball. For instance, the synthetic metal system may comprise a portion which is detectable by a detector or scanner, or the like. In this instance, it is envisaged that a user may use a detector to detect where the ball is. For instance, the synthetic metal system may comprise a unique identifier which may be used by the manufacturer to identify infringing products or for a user to identify they have purchased a genuine product.

In some embodiments of the invention, the synthetic metal system may form substantially the entire ball. In other embodiments of the invention, the synthetic metal system may be provided with one or more layers of material to form the ball. In this instance, it is envisaged that the one or more layers of material may comprise materials typically found in a ball of that type. For instance, where the synthetic metal system forms a portion of the core of a golf ball, the synthetic metal system may be provided with one or more intermediate or mantle layers of materials (such as, but not limited to rubber, synthetic rubber, polybutadiene, resin, thread, yarn, or the like) and one or more outer or cover layers (such as, but not limited to, a thermoplastic resin, ionomer resin, urethane, or the like). For instance, where the synthetic metal system forms a portion of the core of a baseball or softball, the synthetic metal system may be provided with one or more intermediate or mantle layers (such as, but not limited to, yarn, string, rubber, synthetic rubber, or the like) and one or more outer or cover layers (such as, but not limited to, leather, rubber, thermoplastic resin, ionomer, polymer casing, or the like).

In an embodiment of the invention, the synthetic metal system may be configured to provide a desired aesthetic appearance. For instance, the synthetic metal system may be configured such that at least a portion of the frame member comprises aligned frame elements, providing the synthetic metal system with the appearance of carbon fibre materials. For instance, the synthetic metal system may be configured such at least a portion of the frame member comprises a plurality of openings forming a Voronoi pattern, a honeycomb pattern, a tessellation pattern, a pattern of repeating units, a pattern of random units, or the like. In use, it is envisaged that the synthetic metal system may provide the desired aesthetic appearance in a three-dimensional configuration.

In some embodiments, the synthetic metal system may be provided with a unique identifier. Any suitable unique identifier may be used. For instance, the unique identifier may be the unique chemometric profile of a component of the synthetic metal system, a micro RFID tag, a printed plastic tag, a fluorescent tag, a radioisotope marker, an isotope marker, a trace element, a rare earth element, a detectable marker DNA, a biomolecule, or the like

The unique identifier may be located in any suitable component of the synthetic metal system. For instance, the unique identifier may be the matrix material, may be added to the matrix material before the synthetic metal system is formed, may be embedded in the matrix material after the synthetic metal system is formed, may be the frame member, may be added to the material the frame member is fabricated from before the frame member is formed, may be attached to the frame member before the synthetic metal system is formed, may be embedded in the matrix material after the synthetic metal system is formed, or any suitable combination thereof.

In an embodiment of the invention, the unique identifier may be the matrix material of the synthetic metal system. Preferably, the unique identifier may be a unique chemometric profile of the matrix material. In this instance, it is envisaged that the matrix material has a unique chemometric profile which can be detected using a suitable analytical technique, such as near-infrared (NIR) spectroscopy, x-ray fluorescence, or the like. In an embodiment of the invention, the unique identifier may be the frame member of the synthetic metal system. For instance, the unique identifier may be the pattern of the frame elements and/or openings in the frame member or any other configuration of the frame member. In use, it is envisaged that the unique identifier may enable identification and monitor for infringement, provide proof of authenticity, track supply chain movements, or the like.

In a second aspect, the invention resides broadly in a method for manufacturing a synthetic metal system, including the steps of:
(a) Placing a frame member fabricated from a conductive material into a mould cavity;
(b) Injecting a matrix material into the mould cavity, wherein the matrix material is configured to at least partially penetrate openings in the frame member such that the frame member is at least partially encased within the matrix material;
(c) Cooling the mould cavity together with the moulded synthetic metal system until the moulded matrix material is cooled below the glass transition temperature of the matrix material; and
(d) Ejecting the moulded synthetic metal system from the mould cavity.

Preferably, the frame member and the matrix material of the second aspect of the invention are the frame member and the matrix material according to the first aspect of the invention. In an embodiment of the invention, the frame member comprises two or more frame member portions, wherein the frame member portions are configured for connection to one another and/or are retained in place relative to one another within the synthetic metal system by the matrix material.

The method for manufacturing a synthetic metal system may use any suitable moulding process. For instance, the moulding process may include cast moulding, microwave casting, resin injection moulding, compression moulding, insert moulding (overmoulding), injection moulding, or the like. In a preferred embodiment of the invention, the method for manufacturing a synthetic metal system comprises an injection moulding technique. Any suitable injection moulding technique may be used. For instance, injection compression moulding, insert moulding, or the like. However, it will be understood that the type of injection moulding technique used may vary depending on a number of factors, such as the desired properties of the synthetic metal system, the type of matrix material being used, and the type of metal system being used. Advantageously, using a frame member comprising a cellular structure including a plurality of openings facilitates the unimpeded flow of the high temperature and pressure matrix material used in such moulding processes without affecting the integrity of the structure of the frame member or causing disruption to regions of the frame member having specific properties.

The method of manufacturing a synthetic metal system according to a second aspect of the invention may be used to fabricate any suitable component, device or system. For instance, the method of the second aspect of the invention may be used to fabricate a sacrificial anode, a component for electrical circuitry applications or other electronic systems, a component for marine use, a component for aeronautical use, a component for use in space or other thermo-vacuum cyclic environments, a pin plug for an electric appliance, or the like. For instance, the method of the second aspect of the invention may be used to fabricate a component, such as a prosthetics, implants, surgical devices or instruments, or the like, for use in medical or dental applications. For instance, the method of the second aspect of the invention may be used to fabricate a light emitting system, liquid heat exchange system, a vibration damping or shock absorbing element, or the like. For instance, the method of the second aspect of the invention may be used to fabricate an article or a device having a desired aesthetic appearance.

For instance, the method of the second aspect of the invention may be used to fabricate a component associated with a well bore. In this instance, the synthetic metal system may be associated with a component such as a well casing, a portion of a pipeline, a bottom hole assembly (such as, but not limited to, drilling equipment, an artificial lift pump system, a gas lift system, a submersible pump, a hydraulic pump system, a jet pump system, a slurry pump, or the like), a portion of a drill string, a portion of a production tubing string, a portion of a sucker rod string, or the like. In particular, the method of the second aspect of the invention may be used to fabricate a component of a sucker rod pump system or an electric submersible pump system.

For instance, the method of the second aspect of the invention may be used to fabricate a projectile. In particular, the method of the second aspect of the invention may be used to fabricate a ballistic projectile.

For instance, the method of the second aspect of the invention may be used to fabricate at least a portion of a ball. In this instance, the synthetic metal system may replace and function as any suitable portion of a core and/or a mantle layer of the ball or may form substantially the entire ball. In particular, the method of the second aspect of the invention may be used to fabricate a golf ball.

It is envisaged that, in use, the method of manufacturing a synthetic metal system may provide a moulded synthetic metal system which may adapted for connection to any suitable device. Preferably, the synthetic metal system may be adapted for removable connection to the device. For instance, the synthetic metal system may be removably connected to, or retained in, to the device by frictional engagement, or by providing complementary screw-threaded portions, press fittings, snap-fit features, male-female connectors, or combinations thereof. For instance, the synthetic metal system may be connected to the device using one or more mechanical fasteners (screws, bolts, rivets, pins, or the like), an adhesive, a heat or chemical treatment, or welded using any suitable technique.

In an embodiment of the invention, the method of manufacturing a synthetic metal system further comprises the step of placing at least a portion of an elongate member in the mould cavity, wherein the frame member at least partially encases the elongate member. In this instance, it is envisaged that the frame member may comprise a cavity or recessed portion extending along at least a portion of an outer surface of the frame member, wherein the size, shape or configuration of the cavity or recess may be complementary to the size, shape or configuration of the elongate member. It is envisaged that, in use, the synthetic metal system may overmould the elongate member forming a strong bond between the synthetic metal system and the elongate member. In an embodiment of the invention, the elongate member may be any suitable portion of a sucker rod pump system or rod string, such as, but not limited to, a sucker rod and/or a pony rod.

Any of the features described herein can be combined in any combination with any one or more of the other features described herein within the scope of the invention.

The reference to any prior art in this specification is not, and should not be taken as an acknowledgement or any form of suggestion that the prior art forms part of the common general knowledge.

### BRIEF DESCRIPTION OF DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIG. 1 illustrates a side view of a frame member according to an embodiment of the present invention.

FIG. 2 illustrates a side view of a synthetic metal system according to an embodiment of the present invention.

FIG. 3 illustrates a side view of a synthetic metal system in the form of a rod guide connected to a sucker rod according to an embodiment of the invention.

FIG. 4 illustrates a perspective view of a synthetic metal system in the form of a combustion engine piston according to an embodiment of the invention.

FIG. 5 illustrates a partial cutaway view of a golf ball comprising a synthetic metal system core according to an embodiment of the invention.

FIGS. 6A-6D illustrate various frame members for use in a core of a ball according to an embodiment of the present invention.

FIGS. 7A and 7B illustrate various frame members for use in fabricating a synthetic metal system projectile according to an embodiment of the invention.

FIGS. 8A and 8B illustrate a frame member for use in fabricating a synthetic metal system projectile in an unassembled state according to an embodiment of the invention.

FIG. 9 illustrates a frame member for use in fabricating a synthetic metal system projectile in an assembled state according to an embodiment of the invention.

FIG. 10 illustrates a synthetic metal system projectile according to an embodiment of the invention.

### DESCRIPTION OF EMBODIMENTS

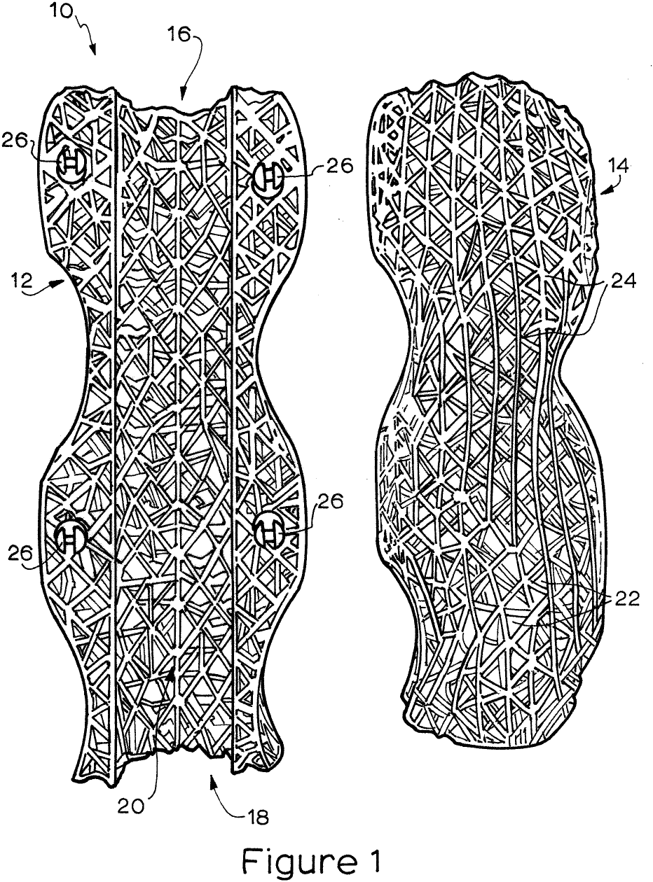

FIG. 1 illustrates a side view of a frame member 10 according to an embodiment of the present invention. In this Figure, frame member 10 comprises two portions 12,14 configured for connection to one another by male-female connection portions 26,28. Frame member 10 comprises a relatively rigid cellular structure comprising a plurality of openings 22 fabricated by three-dimensional printing of a metallic material, wherein the plurality of openings 22 is defined by a lattice of bars 24 laid down during fabrication of the frame member 10.

Frame member 10 is provided with a recessed portion 20 extending from a first end 16 to a second end 18 of the frame member 10, wherein the recessed portion 20 is configured to receive at least a portion of an elongate member (not shown) therein, wherein, when the frame member portions 12,14 of the frame member 10 are connected to one another, it is envisaged that the frame member 10 at least partially encases the elongate member (not shown). The frame member 10 is substantially the same size, shape and configuration of a synthetic metal system suitable for replacing a component in a rod string.

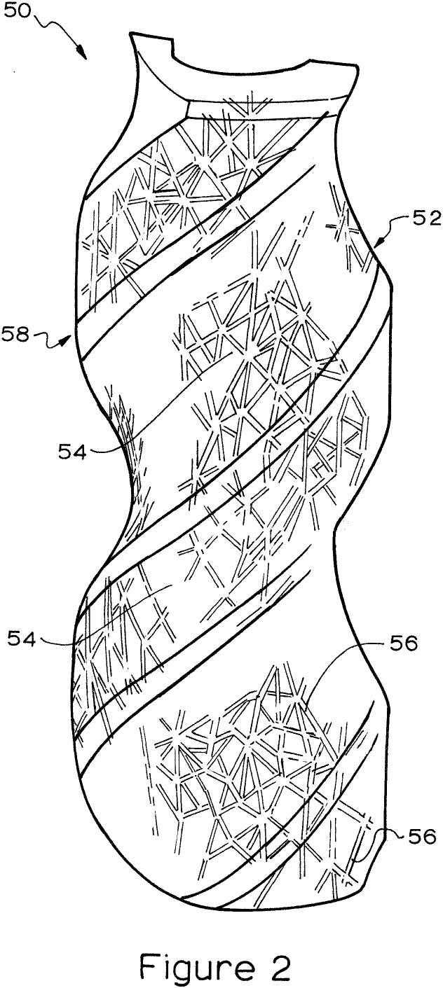

FIG. 2 illustrates a side view of a synthetic metal system 50 according to an embodiment of the present invention. In this Figure, the synthetic metal system 50 comprises a frame member 52 comprising a cellular structure including a plurality of openings and a matrix material 58, wherein the matrix material 58 is configured to at least partially penetrate one or more of the plurality of openings 54 in the frame member 52 such that the frame member 52 is at least partially encased within the matrix material 58.

In use it is envisaged that synthetic metal system 50 may be formed by placing a frame member 52 into a mould cavity (not shown), injecting a matrix material 58 into the mould cavity, wherein the matrix material 58 is configured to at least partially penetrate openings 54 in the frame member 52 such that the frame member 52 is at least partially encased within the matrix material 58, cooling the mould cavity together with the moulded synthetic metal system 50 until the moulded matrix material 58 is cooled below its glass transition temperature and ejecting the moulded synthetic metal system 50 from the mould cavity.

FIG. 3 illustrates a side view of a synthetic metal system 100 in the form of a rod guide 102 connected to a portion of a sucker rod 104 according to an embodiment of the invention. In this Figure, the synthetic metal system 100 may be overmoulded to at least a portion of the sucker rod 104 such that the synthetic metal system 100 may be integrally moulded to at least a portion of the sucker rod 104. Preferably, synthetic metal system 100 comprises a frame member fabricated from an electrically conductive metallic material and a matrix material comprising a polymeric material having low-friction (self-lubricating) and high abrasion resistance.

In use, it is envisaged that the synthetic metal system 100 may replace a rod guide of a rod string, such that the synthetic metal system may replace and function as a rod guide without affecting the operation of a sucker rod pump system comprising the rod string. In use, it is envisaged at least a portion of the frame member may be exposed to the environment through the matrix material, thereby acting as a conduit of electrons and providing cathodic protection to the sucker rod pump system. Similarly, the synthetic metal system may act as a conduit of electrons and provide cathodic protection to any well bore or component thereof, such as a well casing, a portion of a pipeline, a bottom hole assembly, a portion of a production tubing string, a portion of a drill string, or the like.

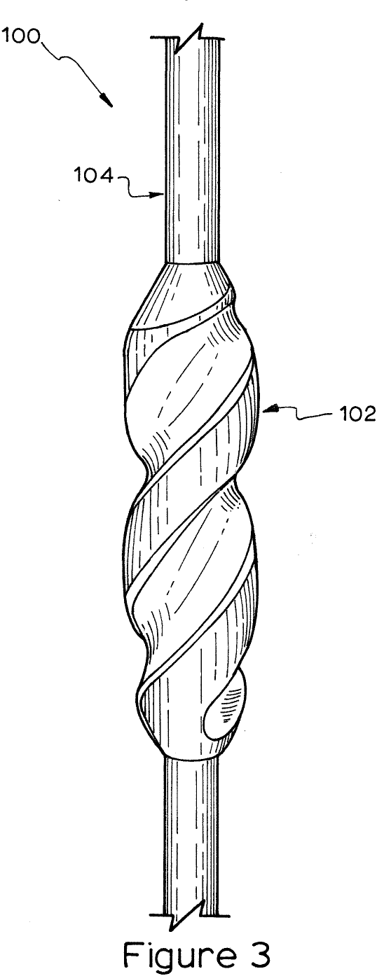

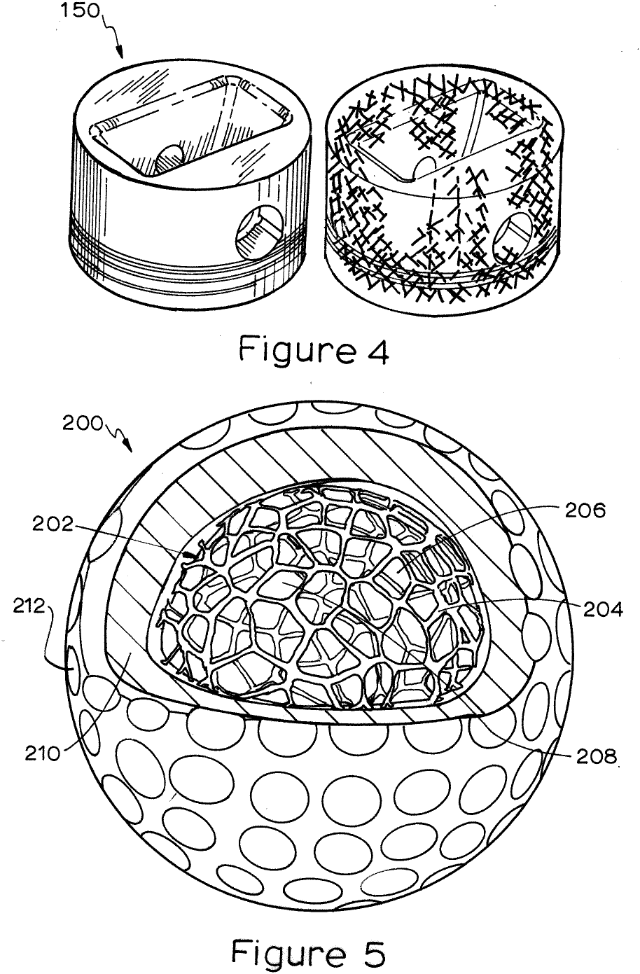

FIG. 4 illustrates a perspective view of a synthetic metal system 150 in the form of a combustion engine piston. In this Figure, the synthetic metal system 150 may comprise an injection moulded ceramic matrix material comprising a steel or steel alloy frame member. In this instance, it is envisaged that the frame member may reduce the brittleness of the ceramic matrix material and may assist in conducting heat away from an outer surface of the combustion engine piston. In this instance, it is envisaged that the frame member may comprise one or more hollow frame elements comprising a coolant or may comprise one or more projections extending out of the matrix material such that heat is removed from the synthetic metal system by convection.

FIG. 5 illustrates a partial cutaway view of a golf ball 200 comprising a synthetic metal system core 202. In this Figure, the synthetic metal system core 202 comprises a frame member 204 and a matrix material 206, wherein the matrix material 206 is configured to at least partially penetrate openings 208 in frame member 204 such that the frame member 204 is at least partially encased within the matrix material 206. Matrix material 206 may be a rubber such as neodymium-catalysed polybutadiene rubber (Nd-BR), a copolymer blend such as ARPMAX®, or the like. Synthetic metal system core 202 of golf ball 200 is provided with an intermediate layer 210 (such as rubber), and an outer cover layer 212 (such as SURLYN® ionomer resin or a polyurethane/acetal blend). In use, it is envisaged that a golf ball comprising a resiliently deformable synthetic metal system core may improve the coefficient of restitution and increasing the moment of inertia, thereby reducing side spin and providing a more stable and predictable flight.

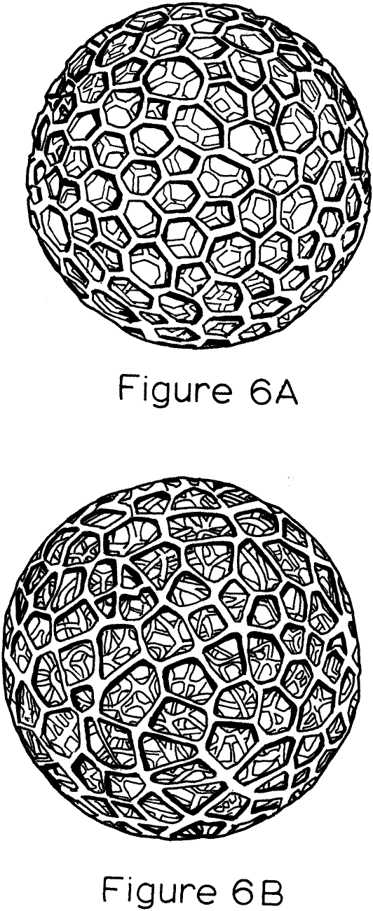

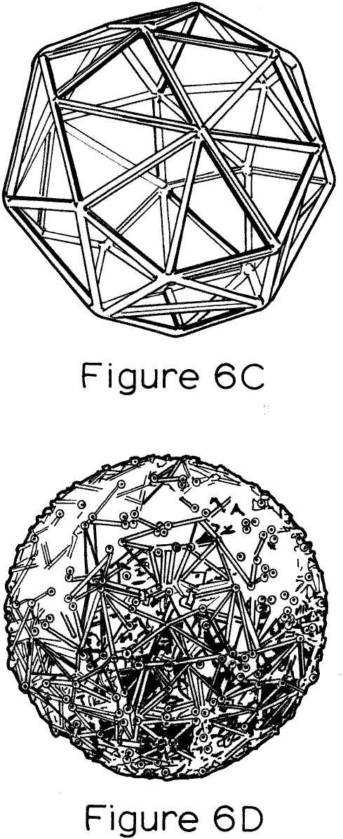

FIGS. 6A-6D illustrate various frame members for use in a synthetic metal system core for a ball. FIGS. 6A and 6B illustrate frame members having a cellular structure wherein the plurality of openings is formed from a Voronoi pattern. FIG. 6C illustrates a frame member having a cellular structure wherein the plurality of openings is triangle-based. FIG. 6D illustrates a frame member having a cellular structure wherein the plurality of openings is a lattice structure comprising a network of nodes and frame elements based on a triangle microstructure.

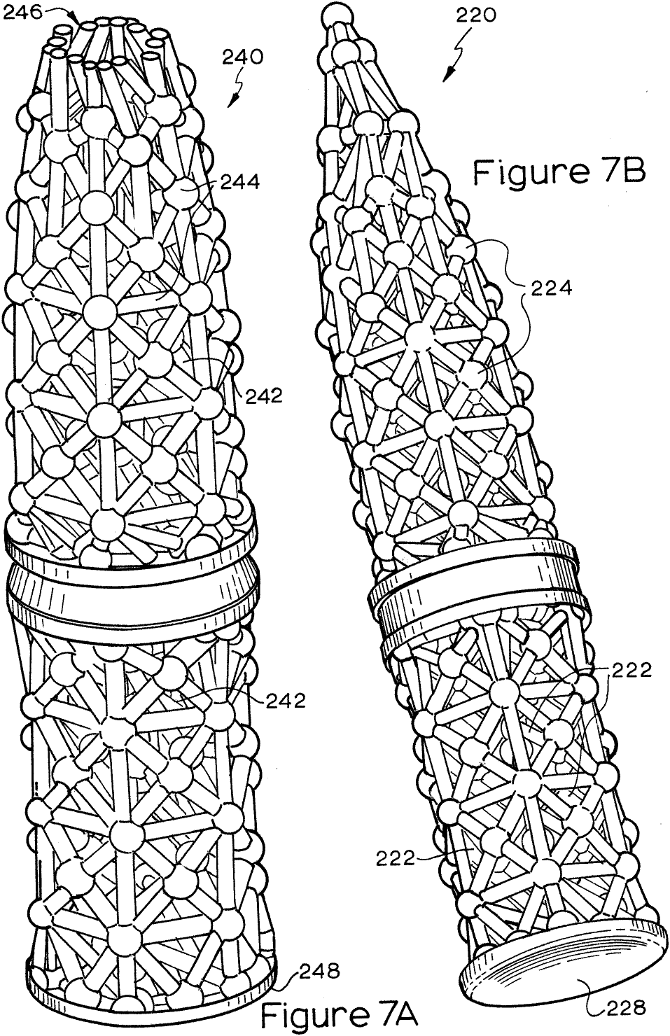

FIGS. 7A and 7B illustrate various frame members for use in fabricating a synthetic metal system projectile. Frame members 220,240 comprises a three-dimensional printed metal alloy lattice structure comprising a plurality of openings 222,242 defined by a plurality of frame elements 224,244. In FIG. 7A, frame member 240 comprises a truncated cone-shaped tip portion 246, wherein the tip of the synthetic metal system projectile may be formed substantially entirely from the matrix material (not shown) during the injection moulding process. The synthetic metal may also form part of the external shell (exoskeleton) which is connected to the endoskeleton lattice at the concave base and/or at the projectile tip. In FIG. 7B, frame member 220 is substantially the same size, shape, and configuration as the synthetic metal system projectile. In FIGS. 5A and 5B, frame member 220,240 may be provided with a base 228,248 having a concave profile configured to direct the explosive energy into the centre of the base of the projectile.

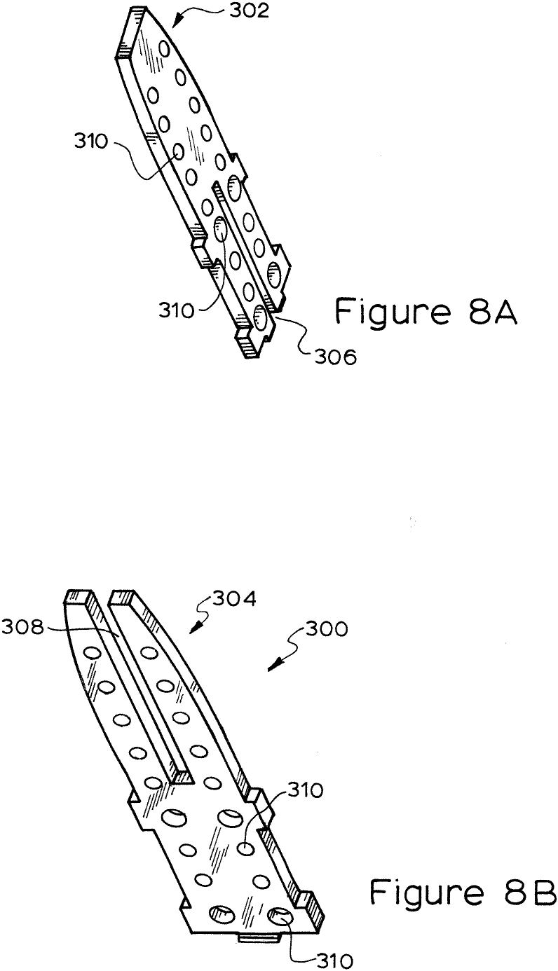

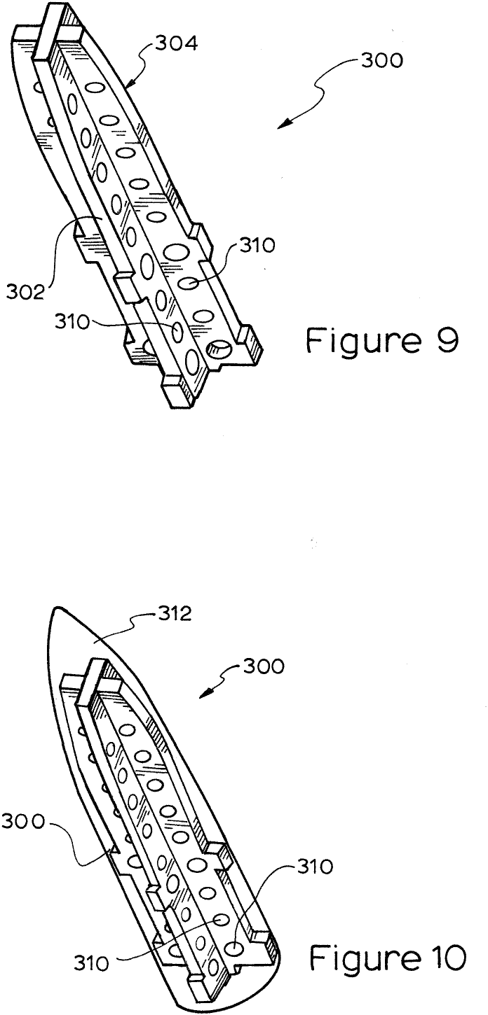

FIGS. 8A-8B, 9 and 10 illustrate a synthetic metal system projectile 300 comprising a frame member fabricated from two portions 302,304. In FIGS. 8A and 8B, a frame member 300 fabricated from two portions 302,304 in a dissembled state is illustrated. Frame member portions 302,304 are configured for connection to one another via complementary elongated slots 306,308 located in a region of each of the frame member portions 302,304. Each frame member portion comprises a plurality of openings 310. In FIG. 9, the frame member 300 is fabricated from two portions 302,304 is illustrated in an assembled state. In FIG. 10, a synthetic metal system comprising a frame member 300 is overmoulded with a matrix material 312, wherein the matrix material is configured to at least partially penetrate openings 310 in the frame member 300 such that the frame member 300 is at least partially encased with the matrix material 312.

In the present specification and claims (if any), the word ‘comprising’ and its derivatives including ‘comprises’ and ‘comprise’ include each of the stated integers but does not exclude the inclusion of one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

1. A synthetic metal system comprising:
a frame member comprising a cellular structure including a plurality of openings;
and a matrix material comprising a polymeric material,
wherein the matrix material is configured to at least partially penetrate one or more of the plurality of openings in the frame member such that the frame member is at least partially encased within the matrix material.

2. The_synthetic metal system according to claim 1 wherein the plurality of openings is defined by a plurality of frame elements which form the frame member.

3. The synthetic metal system according to claim 1 wherein the frame member is a lattice structure.

4. The synthetic metal system according to claims 1 wherein the plurality of openings defines a cavity within and/or on a surface of the frame member.

5. The synthetic metal system according to claim 1 wherein the frame member comprises two or more portions and wherein the frame member portions are configured for connection to one another and/or are retained in place relative to one another within the synthetic metal system by the matrix material.

6. The synthetic metal system according to claim 1, wherein at least a portion of the frame member is in at least partial contact with the environment external to the synthetic metal system.

7. The synthetic metal system according to claim 1, wherein at least a portion of the frame member is fabricated from a metallic material.

8. The synthetic metal system according to claim 1, wherein at least a portion of the frame member is fabricated from an electrically conductive material and/or a thermally conductive material.

9. The synthetic metal system according to claims 1, wherein at least a portion of the frame member is fabricated from a resiliently deformable material.

10. The synthetic metal system according to claim 1, wherein the matrix material comprises one or more thermoplastic, thermosetting and/or elastomeric polymeric materials.

11. The synthetic metal system according to claim 1, wherein the matrix material comprises a polymeric material suitable for processing by injection moulding and/or extrusion.

12. The synthetic metal system according to claims 1, wherein the matrix material comprises a multi-phase chemo-synthetic copolymer thermoplastic polyurethane matrix.

13. The synthetic metal system according to claims 1, wherein the matrix material further comprises a carbon fiber.

14. The synthetic metal system according to claim 1, further comprising a unique identifier.

15. The synthetic metal system according to claims 1, wherein the synthetic metal system is used to fabricate a component of a bottom hole assembly.

16. The synthetic metal system according to claim 15, wherein the synthetic metal system provides cathodic protection to the bottom hole assembly.

17. The synthetic metal system according to claim 1, wherein the synthetic metal system is used to fabricate a core and/or a mantle layer of a ball.

18. The synthetic metal system according to claim 17, wherein the synthetic metal system is resiliency deformable.

19. The synthetic metal system according to claim 1 wherein the synthetic metal system is used to fabricate a projectile.

20. The synthetic metal system according to claims 1 wherein the synthetic metal system used to fabricate a component for use in a medical application and/or a dental application.

21. The synthetic metal system according to claim 20, wherein the synthetic metal system is used to fabricate a prosthetic device and/or an implant.

22. The synthetic metal system according to claim 1 wherein the synthetic metal system is used to fabricate a component for use in space or other thermo-vacuum cyclic environments.

23. A method of manufacturing a synthetic metal system, including the steps of:
a. Placing a frame member comprising a cellular structure including a plurality of openings into a mould cavity;
b. Injecting a matrix material comprising a polymeric material into the mould cavity, wherein the matrix material is configured to at least partially penetrate one or more of the plurality of openings in the frame member such that the frame member is at least partially encased within the matrix material;
c. Cooling the mould cavity together with the moulded synthetic metal system until the moulded matrix material is cooled below the glass transition temperature of the matrix material; and
d. Ejecting the synthetic metal system from the mould cavity.

24. The method of manufacturing a synthetic metal system according to claim 23 wherein the frame member comprises two or more frame member portions and wherein the step of placing the frame member into the mould cavity comprises connecting the respective frame member portions to one another to form the frame member before placing the frame member into the mould cavity.

25. The method of manufacturing a synthetic metal system according to claim 24 wherein the frame member comprises two or more frame member portions and wherein the step of placing the frame member into the mould cavity comprises placing the two or more frame member portions into the mould cavity.

26. The method of manufacturing a synthetic metal system according to claim 23 further comprising the step of placing at least a portion of an elongate member in the mould cavity, wherein the frame member at least partially encases the elongate member.
